(19) United States
(12) Patent Application Publication
(10) Pub. No.: US 2024/0246069 A1
(43) Pub. Date: Jul. 25, 2024

(54) SYSTEMS AND METHODS FOR WET AIR OXIDATION REGENERATION OF CATALYSTS WITH ATMOSPHERIC SWITCHING

(71) Applicant: Virent, Inc., Madison, WI (US)

(72) Inventors: Edgar Steenwinkel, Madison, WI (US); Matt Van Straten, Madison, WI (US); Paul Blommel, Madison, WI (US); Colin Anson, Madison, WI (US); George Tyson, Madison, WI (US); Dana M. Hatch, Madison, WI (US)

(21) Appl. No.: 18/416,818

(22) Filed: Jan. 18, 2024

Related U.S. Application Data
(60) Provisional application No. 63/480,607, filed on Jan. 19, 2023.

(51) Int. Cl. B01J 38/18; B01J 23/46

(52) U.S. Cl. CPC B01J 38/18; B01J 23/462

(57) ABSTRACT

The present disclosure provides systems and methods for producing a regenerated hydrogenation catalyst used for hydrogenating a biomass feedstock. The method can include a regeneration cycle, in which a fouled hydrogenation catalyst is contacted with (a) a first flushing medium comprising water and a gaseous phase comprising oxygen and (b) a second flushing medium comprising water and a gaseous phase comprising at least 90% nitrogen by volume. In particular, multiple regeneration cycles can be used to improve efficiency in removing sulfur-containing impurities from the fouled catalyst.

### CROSS-REFERENCE TO RELATED APPLICATIONS

This application claims priority to U.S. Provisional Application No. 63/480,607, filed Jan. 19, 2023, the content of which is hereby incorporated by reference in its entirety.

### BACKGROUND

Biomass is one category of possible renewable alternatives to petroleum-based fuels, chemicals, and other products. However, it remains challenging to develop efficient, cost effective, and environmentally benign technologies for converting biomass to useful products.

As a promising approach, bioreforming processes provide liquid fuels and chemicals derived from the cellulose, hemicellulose and lignin found in plant cell walls. For instance, cellulose and hemicellulose can be used as feedstock for various bioreforming processes, including aqueous phase reforming (APR) and hydrodeoxygenation (HDO)-catalytic reforming processes that, when integrated with hydrogenation, can convert cellulose and hemicellulose into hydrogen and hydrocarbons, including liquid fuels and other chemical products. APR and HDO methods and techniques are described in U.S. Pat. Nos. 6,699,457; 6,964,757; 6,964,758; and 7,618,612 (all to Cortright et al., entitled “Low-Temperature Hydrogen Production from Oxygenated Hydrocarbons”); U.S. Pat. No. 6,953,873 (to Cortright et al., entitled “Low-Temperature Hydrocarbon Production from Oxygenated Hydrocarbons”); and U.S. Pat. Nos. 7,767,867 and 7,989,664 and U.S. Application No. 2011/0306804 (all to Cortright, entitled “Methods and Systems for Generating Polyols”). Various APR and HDO methods and techniques are described in U.S. Pat. Nos. 8,053,615; 8,017,818; 7,977,517; 8,362,307; 8,367,882; 8,455,705; and 8,933,281 (all to Cortright and Blommel, entitled “Synthesis of Liquid Fuels and Chemicals from Oxygenated Hydrocarbons”); U.S. Pat. No. 8,231,857 (to Cortright, and entitled “Catalysts and Methods for Reforming Oxygenated Compounds”); U.S. Pat. No. 8,350,108 (to Cortright et al., entitled “Synthesis of Liquid Fuels from Biomass”); International Patent Application Publication No. WO 2008/109877 (to Cortright and Blommel, entitled “Synthesis of Liquid Fuels and Chemicals from Oxygenated Hydrocarbons”); all of which are incorporated herein by reference.

In certain applications, it may be beneficial for biomass feedstock to be hydrogenated to increase the thermal stability of the biomass feedstock prior to use as a feed for APR and/or HDO. At temperatures compatible with APR and/or HDO, sugars are susceptible to thermal degradation, which leads to byproduct formation, catalyst fouling, and, ultimately, shortened time between catalyst regenerations. This problem is avoided by reacting sugars with hydrogen to form polyols or sugar alcohols that are more thermally stable.

Biomass feedstock includes impurities, such as sulfur-containing moieties, that poison hydrogenation catalysts over time. Poisoning of the catalyst leads to lower conversion and yield of polyol and sugar alcohol products. As a result, most industrial applications involve a batch or semi-continuous process that involves changing the spent catalyst with fresh catalyst or regenerating the existing catalyst to improve conversion. Changing the hydrogenation catalyst frequently is time consuming, expensive, and can lead to production downtime. Significantly, the poisoning or deactivation of the hydrogenation catalyst is worsened where the feedstock includes elevated sulfur-containing impurities.

Current methods for regenerating hydrogenation catalysts include using multiple hydrogen peroxide washes to remove impurities from the spent hydrogenation catalyst. However, hydrogen peroxide damages the physical strength of the catalyst over time, reducing both total surface area and, ultimately, catalytic activity. There remains a need for more effective, less damaging regeneration systems and methods that restore the catalytic capacity of the hydrogenation catalysts under industrial production conditions.

### SUMMARY OF THE INVENTION

Described herein are reactor systems and methods for regenerating hydrogenation catalysts for use in hydrogenating feedstock solutions, such as water-soluble sugars derived from biomass and/or unsaturated hydrocarbon streams. The provided reactor systems and methods offer unique features and advantages over existing regeneration technologies. This is an improvement over current technologies to regenerate catalytic activity, such as hydrogen peroxide-based methods, which have a tendency to degrade the catalyst's surface area and pore structure over time. Further, hydrogen peroxide poses storage challenges on a commercial scale. The regenerative oxidants provided herein are cheaper than hydrogen peroxide and can be stored at commercial scale using existing technology.

In one aspect, the present disclosure provides a method for hydrogenating biomass. The metho may comprise catalytically reacting a feedstock stream comprising water and an oxygenated hydrocarbon (C₂₊O₁₊) with hydrogen in the presence of a hydrogenation catalyst for a hydrogenation duration to produce a first hydrogenated product stream and a fouled hydrogenation catalyst. The method may further comprise subjecting the fouled hydrogenation catalyst to a regeneration cycle to produce a regenerated hydrogenation catalyst. The regeneration cycle can include: (a) contacting the catalyst with a first flushing medium for an air-treatment duration to produce an air-treated catalyst, wherein the first flushing medium comprises, measured at 25° C. and 1 atmospheric pressure, liquid water and a gaseous phase comprising oxygen, and (b) contacting the air-treated catalyst with a second flushing medium for a nitrogen-treatment duration to produce the regenerated hydrogenation catalyst, wherein the second flushing medium comprises, measured at 25° C. and 1 atmospheric pressure, liquid water and a gaseous phase comprising at least 90% nitrogen by volume. The method may further comprise catalytically reacting the feedstock stream with hydrogen in the presence of the regenerated hydrogenation catalyst to further produce a second hydrogenated product stream.

In another aspect, the present disclosure provides a method for producing a regenerated hydrogenation catalyst from a fouled hydrogenation catalyst. The method may comprise catalytically reacting a feedstock stream having at least one sulfur-containing impurity in the presence of a hydrogenation catalyst to produce the fouled hydrogenation catalyst, wherein the fouled hydrogenation catalyst comprises an amount of sulfur derived from the at least one sulfur-containing impurity of the feedstock stream. The method may further comprise subjecting the fouled hydrogenation catalyst to a regeneration cycle to produce a regenerated hydrogenation catalyst. The regeneration cycle can include: (a) contacting the catalyst with a first flushing medium for an air-treatment duration to produce an air-treated catalyst, wherein the first flushing medium comprises, measured at 25° C. and 1 atmospheric pressure, liquid water and a gaseous phase comprising oxygen, and (b) contacting the air-treated catalyst with a second flushing medium for a nitrogen-treatment duration to produce a nitrogen-treated catalyst, wherein the second flushing medium comprises, measured at 25° C. and 1 atmospheric pressure, liquid water and a gaseous phase comprising at least 90% nitrogen by volume. In particular, the amount of sulfur in the nitrogen-treated hydrogenation catalyst is reduced relative to the fouled hydrogenation catalyst.

In some embodiments, the present method comprises subjecting the nitrogen-treated catalyst to a successive set of the operations (a) and (b) of the regeneration cycle, each with a respective treatment duration, to produce the regenerated hydrogenation catalyst. For example, the conditions of one regeneration cycle (such as temperature, pressure, and duration) may be controlled independent of the corresponding conditions of another regeneration cycle.

In various embodiments, the hydrogenation catalysts of the present methods may include ruthenium on carbon (Ru/C).

### BRIEF DESCRIPTION OF DRAWINGS

FIG. 1 shows an example reactor system in accordance with some embodiments of the present disclosure.

FIG. 2 shows representative results of sulfur content analysis of effluent samples from a wet air oxidation regeneration (WAOR) process on a 2 wt % Ru/C catalyst. Six samples were taken while air was flowing through reactor (BDL=below detection limit), one sample was taken while N₂ is purging, and one final sample was taken during wet reduction.

FIG. 3 shows comparison of four regeneration processes (Regens 1-4) on a Ru/C catalyst. Regens 1-3 all ran air for 24 hours, while Regen 4 ran air for 4 hours.

FIG. 4 shows performance of a Ru/C catalyst as characterized by conversion and hydrogenation reactor inlet temperature. Regenerations are marked with vertical lines, and changes in corn syrup batches are shown in gray dashed lines.

FIG. 5 shows the sulfur content, conductivity, and pH in the effluent from a “pulsed” WAOR process, in which the reactor is switched between air flow and N₂ flow every 12 hours.

FIG. 6 shows the sulfur content and conductivity of effluents from a second pulsed regeneration process on a Ru/C catalyst.

FIG. 7 shows the sulfur content and conductivity data of a pulsed regeneration process using 30-minute air flow (pulses) on a Ru/C catalyst.

### DETAILED DESCRIPTION OF THE INVENTION

Described herein are reactor systems and methods for regenerating hydrogenation catalysts for use in hydrogenating feedstock solutions, such as water-soluble sugars derived from biomass and/or unsaturated hydrocarbon streams. The provided reactor systems and methods offer unique features and advantages over existing regeneration techniques. A wet air oxidation regeneration system was disclosed in the co-pending U.S. patent application Ser. No. 17/891,093, the content of which is incorporated herein by reference in its entirety, which offers mild reaction conditions that can effectively remove impurities to restore hydrogenation catalytic activity, while additionally maintaining the catalyst's structural integrity (e.g., surface area, pore volume). Maintaining the catalyst's structural integrity and/or catalytic activity for extended periods of time improves operation economics by reducing the number of times the catalyst needs to be replaced over time and/or reducing the required frequency of regeneration operations. The present regeneration system retains the advantages of the system disclosed in U.S. Ser. No. 17/891,093. Remarkably, the present system is adapted to atmospheric switching (e.g., from air to nitrogen) during the regeneration process and offers more efficient regeneration results as well as the additional advantage of effectively regenerating catalysts used in hydrogenating feedstocks with a high level of sulfur-containing impurities.

In one aspect, the present disclosure provides method for hydrogenating biomass, the method comprising:
catalytically reacting a feedstock stream comprising water and an oxygenated hydrocarbon (C₂₊O₁₊) with hydrogen in the presence of a hydrogenation catalyst for a hydrogenation duration to produce a first hydrogenated product stream and a fouled hydrogenation catalyst;
subjecting the fouled hydrogenation catalyst to a regeneration cycle to produce a regenerated hydrogenation catalyst, the regeneration cycle comprising:
   (a) contacting the catalyst with a first flushing medium for an air-treatment duration to produce an air-treated catalyst, wherein the first flushing medium comprises, measured at 25° C. and 1 atmospheric pressure, liquid water and a gaseous phase comprising oxygen, and
   (b) contacting the air-treated catalyst with a second flushing medium for a nitrogen-treatment duration to produce the regenerated hydrogenation catalyst, wherein the second flushing medium comprises, measured at 25° C. and 1 atmospheric pressure, liquid water and a gaseous phase comprising at least 90% nitrogen by volume; and
catalytically reacting the feedstock stream with hydrogen in the presence of the regenerated hydrogenation catalyst to further produce a second hydrogenated product stream.

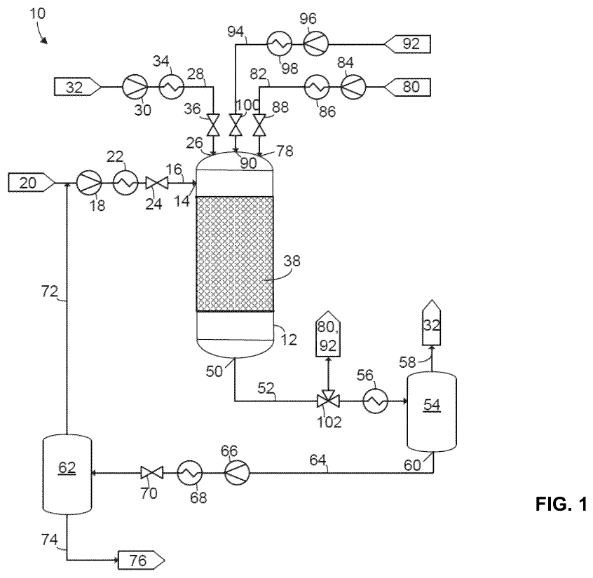

The present method may be carried out in a reactor system. Referring to FIG. 1, a representative reactor system 10 is illustrated in accordance to some embodiments of the present disclosure. Although the principles disclosed herein can be beneficially implemented on the illustrated reactor system 10, use of other reactor system architectures is possible for some embodiments. In particular, the reactor system 10 includes a reactor 12 having a feedstock inlet 14 that places the reactor 12 in fluid communication with a feedstock conduit 16. A pump 18 may be configured in the feedstock conduit 16 to transport a feedstock solution from a feedstock source 20, such as a reservoir or upstream process unit, to the reactor 12. The feedstock conduit 16 may include a heat exchanger 22 for controlling the temperature of the feedstock solution, and a valve 24 for controlling the flow of the feedstock solution to the reactor 12.

In some embodiments, suitable feedstock solutions include water-soluble sugars derived from biomass, although other feedstocks can be used. As used herein, the term “biomass” refers to, without limitation, organic materials produced by plants (such as leaves, roots, seeds and stalks), and microbial and animal metabolic wastes. Common biomass sources include: (1) agricultural wastes, such as corn stalks, straw, seed hulls, sugarcane leavings, bagasse, nutshells, and manure from cattle, poultry, and hogs; (2) wood materials, such as wood or bark, sawdust, timber slash, and mill scrap; (3) municipal waste, such as waste paper and yard clippings; and (4) energy crops, such as poplars, willows, switch grass, alfalfa, prairie bluestream, corn, soybean, and the like. The feedstock can be fabricated from biomass by any means now known or developed in the future, or can be simply byproducts of other processes. The sugars can also be derived from wheat, corn, sugar beets, sugar cane, or molasses. The sugar is combined with water to provide an aqueous feedstock solution having a concentration effective for hydrogenating the sugar. Generally, a suitable sugar concentration is in the range of about 5% to about 70%, with a range of about 40% to 70% more common in industrial applications.

Additionally or alternatively, suitable feedstock solutions include, but are not limited to, oxygenated hydrocarbons (C₂₊O₁₊, e.g. cyclic ethers, esters, ketones, lactones, carboxylic acids), vegetable oils (e.g., polyunsaturated fatty acids), olefins (e.g., alkenes and aromatics, such as C₃-C₁₂ olefins), alkynes, aldehydes, imines, nitriles, thiols, disulfides, thioesters, thioethers, phenols, other arenes/aromatic compounds, and combinations thereof. In some embodiments, the feedstock stream comprising water and an oxygenated hydrocarbon (C₂₊O₁₊). In some embodiments, the oxygenated hydrocarbon is a saccharide.

Referring back to FIG. 1, the reactor 12 includes a hydrogen inlet 26 that places the reactor 12 in fluid communication with a hydrogen conduit 28. A gas transport device 30 may be configured in the hydrogen conduit 28 to transport hydrogen from hydrogen source 32, such as a reservoir or upstream process unit, to the reactor 12. In some embodiments, the hydrogen conduit 28 includes a heat exchanger 34 configured to control the heat of the hydrogen stream. Suitable gas transport devices 30 include, but are not limited to, compressors or blowers. Although the hydrogen inlet 26 and the feedstock inlet 14 are orientated in a co-current direction in FIG. 1, it is to be appreciated that the hydrogen inlet 26 could be arranged in a countercurrent orientation (i.e., fed into the bottom of the reactor 12). The hydrogen conduit 28 may include a valve 36 for controlling the flow of the hydrogen to the reactor 12. Although not illustrated in FIG. 1, the feedstock and hydrogen may be blended, mixed, or otherwise combined in a mixer prior to being delivered to the reactor 12.

In some embodiments, the reactor 12 includes a hydrogenation catalyst 38 disposed therein. Hydrogenation reactions can be carried out in any reactor of suitable design, including continuous-flow, batch, semi-batch or multi-system reactors, without limitation as to design, size, geometry, flow rates, etc. The reactor system 10 can also use a fluidized catalytic bed system, a swing bed system, a fixed bed system, a moving bed system, or a combination of the above. Reactions of the present disclosure are typically practiced using a continuous flow system at steady-state equilibrium.

In some embodiments, the reactor system 10 operates as a fixed, trickle bed reactor with shell-and-tube heat exchange in which the hydrogen and feedstock solution are introduced at the top of the reactor 12 and allowed to flow downward over a fixed bed of the hydrogenation catalyst 38. The advantages of the trickle bed reactor include a simple mechanical design, a simplified operation and potentially a simplified catalyst development. The main design challenges are ensuring that the heat and mass transfer requirements of the reaction are met. The main operational challenges for trickle bed reactors are: uniformly loading the hydrogenation catalyst 38, uniformly introducing the gas and liquid feeds, and avoiding bypassing of some of the hydrogenation catalyst 38 due to channeling of the reactants as they flow through the reactor 12.

In some embodiments, the reactor system 10 operates as a slurry reactor. While a trickle bed reactor is loaded with an immobile hydrogenation catalyst 38, a slurry reactor contains a flowing mixture of reactants, products, and hydrogenation catalyst 38 particles. Keeping a uniform mixture throughout the reactor 12 includes active mixing either from a mixer or a pump. In addition, to withdraw product the catalyst particles must be separated from the product and unreacted feed by filtration, settling, centrifuging or some other means. The advantages of a slurry reactor are mainly that the active mixing might enable higher heat and mass transfer rates per unit of reactor volume.

In some embodiments, the feedstock solution and the hydrogen are reacted across the hydrogenation catalyst 38 in the reactor 12. In some embodiments, the heat exchangers 22, 34 heat the feedstock solution and hydrogen streams to a temperature from 5° C. to 700° C., from 10° C. to 500° C., from 20° C. to 300° C., or from 50° C. to 180° C. In some embodiments, the pressure of the reactor 12 is maintained from 0 psig to 5000 psig, or from 100 psig to 3000 psig. The hydrogenation catalyst 38 may be configured in the reactor 12 in various configurations including, but not limited to, a single fixed bed or in a shell and tube arrangement. In some embodiments, the reactor system 10 includes a heating system configured to provide heat to the reactor 12 to maintain a desired operating temperature. In some embodiments, the heating system provides heat to the reactor 12 using, for example, a heating element (e.g., electric heaters), a heating fluid, or combinations thereof. The heating system may be configured on the outside of the reactor. Additionally or alternatively, the heating system may be configured in a shell-and-tube configuration, where a heating fluid provides heat to the hydrogenation catalyst 38 via the shell or tube side. In some embodiments, the reactor 12 temperature can also be controlled by recycling the products of the reaction back through the reactor 12 to decrease the reaction exotherms.

The product stream exits the reactor 12 through at least one reactor outlet 50 and is optionally transported to a separator 54 via a product conduit 52. In some embodiments, the product conduit 52 includes a heat exchanger 56 to adjust the temperature of the product stream prior to entering the separator 54. The separator 54 may optionally separate unreacted hydrogen from unreacted reactants and products. The unreacted hydrogen may be recycled to the hydrogen source 32 via a hydrogen recycle conduit 58. Any suitable separator 54 may be used to separate the hydrogen from the unreacted reactants and products, including but not limited to, a settling tank, flash tank, distillation, or a combination thereof. Although not illustrated in FIG. 1, in some embodiments the reactor 12 may include a gas outlet and a liquid outlet, where the disengagement of vapor and liquid products occurs inside the reactor 12 without the separator 54.

In some embodiments, the separator 54 includes a product outlet 60 that places the separator 54 in fluid communication with a second separator 62 via conduit 64. A pump 66 may transport the product stream and unreacted reactants to the second separator 62. A heat exchanger 68 may control the temperature of the product stream and unreacted reactants entering the second separator 62, and a valve 70 may regulate the flow.

In some embodiments, the second separator 62 is configured to separate the product stream from unreacted reactants. The unreacted reactants may be recycled to the feedstock conduit 16 via recycle conduit 72, or otherwise discarded from the process. The product stream exiting the separator 62 via product conduit 74 may be sent to storage or to downstream processing units 76, such as aqueous phase reforming (APR) or hydrodeoxygenation (HDO) systems. Any suitable separator 62 may be used to separate the product stream from the unreacted reactants, including but not limited to, distillation, evaporation, liquid-liquid extraction, chromatography, or combinations thereof.

### Catalyst

The present method may be used for regenerating hydrogenation catalysts, e.g., those used in the hydrogenation of biomass. In some embodiments, suitable hydrogenation catalysts 38 for the reactor system 10 includes hydrogenation catalysts 38 having an active metal and a support. Suitable active metals include, but are not limited to, Fe, Ru, Co, Pt, Pd, Ni, Re, Cu, alloys thereof, and a combination thereof, either alone or with promoters such as Ag, Au, Cr, Zn, Mn, Mg, Ca, Cr, Sn, Bi, Mo, W, B, P, and alloys or combinations thereof.

The hydrogenation catalyst may also include any one of several supports, depending on the desired functionality of the catalyst. Exemplary supports include transition metal oxides, an oxide formed from one or more metalloid, and reactive nonmetals (e.g., carbon). Non-limiting examples of supports include, but are not limited to, carbon, silica, alumina, zirconia, titania, vanadia, ceria, silica-aluminate, zeolite, kieselguhr, hydroxyapatite, zinc oxide, chromia, and mixtures thereof.

In some embodiments, the catalyst is a ruthenium on carbon (Ru/C) hydrogenation catalyst. In some embodiments, the catalyst comprises about 0.1% to about 5% by weight ruthenium loaded onto a carbon particle, which includes, but is not limited to, about 0.5%, 1.0%, 1.1%, 1.2%, 1.3%, 1.4%, 1.5%, 1.6%, 1.7%, 1.8%, 1.9%, 2.0%, 2.5%, 3.0%, 3.5%, 4.0%, and 4.5% by weight. In some embodiments, the catalyst comprises about 0.1% to about 4.0% or about 1.0% to about 2.0% by weight ruthenium on carbon. The catalyst may be in a form of extrudate, tablet, sphere, granule, powder, foam, a coated structure, or a combination thereof.

The catalyst may be deactivated during the reaction or chemical process it catalyzes. For example, a hydrogenation catalyst as described herein may be deactivated during biomass hydrogenation process. The catalyst may have a surface with active sites, which may affect the capacity of the catalyst in catalyzing the hydrogenation reaction. The catalyst may be deactivated due to various reasons during the hydrogenation process, including, for example, blocking of active sites by physical absorption (or deposition) of bulky molecules, poisoning of active sites by impurities in the feedstock, or a combination thereof. Catalyst poisoning may be caused by, for example, a chemical reaction or strong interaction of the impurities (e.g., sulfur containing compounds) with the active site of the catalyst, thereby lowering the capacity of the catalyst to catalyze the hydrogenation reaction, i.e., thereby deactivating the catalyst. The degree of deactivation of the catalyst may increase over time as the hydrogenation process continues. Although the amounts of impurities in the feedstock may be relatively low, at large volumes and over time the impurities can build up and adversely affect catalyst activity.

A “fresh” catalyst is used to mean a catalyst that has not been exposed to a feedstock solution or the impurities from the feedstock under hydrogenation conditions.

A “fouled hydrogenation catalyst” or “fouled catalyst” as used herein refers to a hydrogenation catalyst in which the active sites are at least partially deactivated due to being used in a hydrogenation process (i.e., exposed to a feedstock solution under conditions for hydrogenation of the feedstock solution using the catalyst). The degree of fouling may be affected, for example, by the composition of the catalyst, the duration and conditions of the hydrogenation process, the composition of the feedstock, and the amounts of impurities in the feedstock.

A “regenerated hydrogenation catalyst” or “regenerated catalyst” as used herein refers to a fouled catalyst whose catalytic capacity is at least partially restored, for example, by removing the deposits and/or accumulated impurities from the catalyst surface, restoring access to active sites, restoring poisoned active sites, or a combination thereof. As described herein, a regenerated catalyst may be re-used in a hydrogenation process and become a fouled catalyst again during the process. In this situation, the regenerated catalyst can also be referred to as a “freshly regenerated” catalyst relative to the fouled catalyst produced from such regenerated catalyst.

The catalytic capacity of a regenerated catalyst, or the catalytic capacity of a fouled catalyst from which the regenerated catalyst is produced, may be compared to that of a fresh catalyst. For example, the catalytic capacity of a fouled catalyst may be about 20%, about 30%, about 40%, about 50%, about 60%, about 70%, about 80%, or about 90% of the catalytic capacity of a fresh catalyst. For example, the catalytic capacity of a regenerated catalyst may be about 80%, about 90%, about 95%, about 99%, about 100%, or about 110% the catalytic capacity of a fresh catalyst. In some embodiments, the regenerated catalyst has more catalytic capacity than the fouled catalyst from which the regenerated catalyst is produced. For example, the regeneration method herein may restore at least a portion of the catalytic capacity in a fouled catalyst, resulting in an increase of the catalytic capacity in the regenerated catalyst. In some embodiments, the catalytic capacity in a regenerated catalyst may be about 105% to about 500% of the catalytic capacity of the fouled catalyst from which the regenerated catalyst is produced, including about 120%, about 150%, about 200%, about 300%, about 400%, or about 500%.

The catalytic capacity of a catalyst (e.g., a fresh, fouled, or regenerated catalyst) can be measured by a conversion rate of a reagent in the feedstock in a reaction (e.g., a hydrogenation reaction) that is catalyzed by such catalyst. As used herein, the term “conversion” of a hydrogenation catalyst refers to the hydrogenation catalyst's conversion over a duration (e.g., at least 1 hour to at least one day) of a reactant in the feedstock solution after being exposed to the feedstock solution for a hydrogenation cycle. The hydrogenation catalyst may be a fresh catalyst, a fouled catalyst, or a regenerated catalyst. As used herein, conversion of a specific feedstock reactant (Xᵢ) may be calculated by:

$$\\ {X}_{i} = 1 - \frac{{n}_{i} ( t )}{{n}_{i} ( t = 0 )}$$

where nᵢ is the number of moles of the specific feedstock reactant (e.g., sugar, olefin, vegetable oil, alkyne, aldehyde, imine, nitrile) at the beginning (t=0) or after a specific duration (t) of the hydrogenation process. The conversion values of a fresh catalyst, a fouled catalyst, and a regenerated catalyst may be compared under the same hydrogenation conditions (e.g., at a temperature from 50° C. to 180° C. and a pressure from 100 psig to 3000 psig), as conversion may be a function of temperature and pressure.

In some embodiments, the reactor system 10 includes pre-treatment units or steps to process the feedstock solution and/or hydrogenation catalyst 38. For example, the hydrogenation catalyst 38 may be reduced into an active state. For example, during production, the catalyst can be reduced and, in certain applications, then passivated with low levels of oxygen to stabilize the catalyst when exposed to air. The purpose of the reduction step is to transform any oxidized catalyst into a fully reduced state. For certain feedstock solutions, a pre-treatment step may be included upstream of the reactor system 10. For example, sugars containing glycosidic bonds (e.g., sucrose) may be hydrolyzed prior to hydrogenation in the reactor 12.

### Catalyst Regeneration

During hydrogenation, catalyst impurities may build up on the surface of the hydrogenation catalyst 38 and reduce catalytic performance. As used herein, the terms “catalyst impurity” or “impurity” refers to impurities that form deposits that accumulate on catalytic sites on the surface of hydrogenation catalyst 38, restrict access to the catalytic sites, and/or reduce catalytic activity over time (i.e., results in lower conversion and yields of products). Exemplary catalyst impurities include, but are not limited to, carbon-containing impurities, sulfur-containing impurities, silicon-containing impurities, phosphorus-containing impurities, or iron-containing impurities.

In some embodiments, the hydrogenation catalyst 38 is regenerated into a regenerated catalyst by contacting the hydrogenation catalyst 38 with a flushing medium. In some embodiments, the flushing medium comprises a vapor or gaseous phase and a liquid phase. Unless otherwise specified, the terms “vapor phase” and “gaseous phase” are used interchangeably herein with regard to the flushing medium, and the physical properties of the “gaseous phase” and “liquid phase” of the flushing medium are understood to be measured at 25° C. and 1 atmospheric pressure. For example, the flushing medium can include, measured at 25° C. and 1 atmospheric pressure, water as the liquid phase and air or nitrogen in the gaseous phase.

Still referring to FIG. 1, the reactor 12 includes a vapor phase inlet 78 that places the reactor 12 in fluid communication with a vapor phase source 80 via vapor phase conduit 82. A fluid transport device 84 (e.g., compressor or blower) may be configured in the vapor phase conduit 82 to transport the vapor phase from the vapor phase source 80 to the reactor 12. The vapor phase conduit 82 may include a heat exchanger 86 for controlling the temperature of the flushing medium's vapor phase, and a valve 88 for controlling the flow of the vapor phase to the reactor 12. In some embodiments, the fluid transport device 84 is configured for direct air or atmospheric capture, where the fluid transport device 84 is in fluid communication or in direct fluid communication with atmospheric air for compression. Using air as the vapor phase in the flushing medium offers various advantages. Specifically, this would avoid having to purchase and store other oxidants (e.g., hydrogen peroxide) on site. In some embodiments, the vapor phase source 80 includes a gaseous phase that comprises oxygen, for example, at about 0.1% to about 30% by volume. In some embodiments, the vapor phase source 80 includes an inert gas (e.g., nitrogen, argon, helium, neon, krypton, xenon, radon, or combinations thereof) source and oxygen source (e.g., compressed tank) that may be used to alter the O₂ and/or inert gas content of the vapor phase to the concentrations described herein. In some embodiments, the vapor phase source 80 includes at least 50% air by volume, such as at least 60%, at least 70%, at least 80%, at least 90%, at least 95%, at least 96%, at least 97%, at least 98%, or at least 99% air by volume. In some embodiments, the vapor phase source 80 includes at least 90% nitrogen by volume, such as at least 95%, at least 96%, at least 97%, at least 98%, or at least 99% nitrogen by volume. By adjusting the types of gases (e.g., air or inert gases) supplied via vapor phase source 80, the oxidative atmosphere of the flushing medium (e.g., as measured by the amount of oxygen in the gaseous phase) can be tailored.

In some embodiments, the reactor 12 includes a liquid phase inlet 90 that places the reactor 12 in fluid communication with a liquid phase source 92 via liquid phase conduit 94. In some embodiments, the liquid phase comprises water. A pump 96 may be configured in the liquid phase conduit 94 to transport the liquid phase from the liquid phase source 92 to the reactor 12. The liquid phase conduit 94 may include a heat exchanger 98 for controlling the temperature of the flushing medium's liquid phase, and a valve 100 for controlling the flow of the vapor phase to the reactor 12. Although not illustrated in FIG. 1, the liquid phase and vapor phase may be blended, mixed, or otherwise combined in a mixer prior to being delivered to the reactor 12.

In some embodiments, the regenerated hydrogenation catalyst may be produced by maintaining contact of the flushing medium with the hydrogenation catalyst 38 at a regeneration temperature, a regeneration pressure, and a duration sufficient to remove at least a portion of the impurities from the hydrogenation catalyst 38. Contacting the flushing medium to the hydrogenation catalyst 38 may occur in any suitable flow scheme, including continuous flow of flushing medium over the hydrogenation catalyst 38 without recycle, continuous flow of flushing medium over the hydrogenation catalyst 38 with some or full recycle, batch, or semi-batch flow. In some embodiments, the flushing medium exits the reactor 12 through reactor outlet 50, and is recycled to the flushing medium sources 80, 92 or reactor inlets 78, 90 by controlling the flow in product conduit 52 with valve 102. The liquid phase (e.g., water) and gaseous phase (e.g., air or nitrogen) of the flushing medium may be recycled several times before being replaced by flesh flushing medium. A “recycle rate” refers to ratio of recycled material (e.g., water) to fresh material. The present system can allow a high recycle rate (e.g., 6, 7, 8, 9, or 10) using water as the liquid phase of the flushing medium (e.g.; a recycle rate of 10 corresponds to 10 equivalents of water being recycled per one equivalent of fresh water being introduced to the system). The high recycle rate can reduce water consumption, which is particularly advantageous on large production scales.

### Regeneration Cycle

In some embodiments, the first flushing medium comprises, measured at 25° C. and 1 atmospheric pressure, liquid water and a gaseous phase comprising at least 0.1% oxygen by volume, including but not limited to at least 0.5%, at least 1%, at least 2%, at least 5%, at least 8%, at least 10%, at least 12%, at least 15%, at least 18%, at least 20%, at least 25%, at least 30%, or at least 35% oxygen by volume. In some embodiments, the oxygen content of the gaseous phase of the first flushing medium is about 0.1% to about 30% by volume, such as about 0.5% to about 30%, about 1% to about 30%, about 1% to about 25%, about 1% to about 20%, or about 5% to about 20% by volume. In some embodiments, the oxygen content of the gaseous phase of the first flushing medium is about 5% to about 20% by volume. In some embodiments, the oxygen content of the gaseous phase of the first flushing medium is about 1%, about 5%, about 10%, about 15%, about 20%, or about 25% by volume. In some embodiments, the oxygen content of the gaseous phase of the first flushing medium is about 20% by volume. The gaseous phase of the first flushing medium can further comprise an inert gas. The inert gas can be, for example, nitrogen, argon, helium, neon, krypton, xenon, radon, carbon dioxide, or a combination thereof.

The gaseous phase of the first flushing medium can have a composition that is the same as or similar to air. In some embodiments, the first flushing medium comprises, measured at 25° C. and 1 atmospheric pressure, liquid water and a gaseous phase comprising at least 50% air by volume, including but not limited to at least 60%, at least 70%, at least 80%, at least 90%, at least 95%, at least 96%, at least 97%, at least 98%, or at least 99% air by volume. In some embodiments, the gaseous phase of the first flushing medium consists of air. As used herein, “air” refers to gases surrounding the earth, which may vary regionally, and are a function of various factors, such as temperature and pressure. As one example, the term “air” may refer to a gaseous composition composed, in a dry volume percentage (vol %), of about 78 vol % nitrogen, about 20.9 vol % oxygen, about 0.9 vol % argon, about 0.04 vol % carbon dioxide, and other elements and compounds such as helium, methane, krypton, hydrogen, nitrous oxide, xenon, ozone, carbon monoxide, sulfur dioxide, nitrogen dioxide, and ammonia. In this example, a gaseous phase comprising at least 50% air by volume would comprise at least 10% oxygen by volume. As nonlimiting examples, the gaseous phase of the first flushing medium can be air, a mixture of air and nitrogen, a mixture of air and oxygen, a mixture of oxygen and nitrogen, or a mixture of oxygen with one or more inert gases. The oxygen and nitrogen contents (net vol %) in some exemplary gaseous phases (made of the supplied gases) of the first flushing medium are as follows.

| Supplied | Supplied | Net Oxygen | Net Nitrogen |
|---|---|---|---|
| Air | Nitrogen | (approx.) | (approx.) |
| 5% | 95% | 1% | 99% |
| 10% | 90% | 2% | 98% |
| 50% | 50% | 10% | 90% |
| 75% | 25% | 15% | 85% |
| 100% | 0% | 20% | 80% |
| Supplied | Supplied | Net Oxygen | Net Nitrogen |
| Air | Oxygen | (approx.) | (approx.) |
| 95% | 5% | 24% | 76% |
| 90% | 10% | 28% | 72% |
| 80% | 20% | 36% | 64% |
| Supplied | Supplied | Net Oxygen | Net Nitrogen |
| Oxygen | Nitrogen | (approx.) | (approx.) |
| 1% | 99% | 1% | 99% |
| 20% | 80% | 20% | 80% |

The present method may include maintaining contact of the hydrogenation catalyst with the first flushing medium for an air-treatment duration to produce an air-treated catalyst. As used herein, the terms “air-treatment,” “air-treated,” “oxygen-treatment,” “oxygen-treated,” and the like, refer to a treatment with air or any other natural or artificial gaseous composition that includes oxygen. The air-treatment duration can be at least 30 minutes, including but not limited to at least 1 hour, at least 2 hours, at least 4 hours, at least 8 hours, at least 12 hours, or at least 24 hours. In some embodiments, the air-treatment duration is about 2 hours to about 8 hours, such as about 2 hours to about 4 hours.

In some embodiments, the second flushing medium comprises, measured at 25° C. and 1 atmospheric pressure, liquid water and a gaseous phase comprising at least 90% nitrogen by volume, including but not limited to at least 95%, at least 96%, at least 97%, at least 98%, or at least 99% nitrogen by volume. In some embodiments, the gaseous phase of the second flushing medium comprises at least 95% nitrogen by volume. In some embodiments, the gaseous phase of the second flushing medium comprises at least 99% nitrogen by volume. In some embodiments, the gaseous phase of the second flushing medium consists of nitrogen. In some embodiments, the gaseous phase of the second flushing medium is essentially free of oxygen. As used herein, the term “essentially free of oxygen” refers to less than 1%, or less than 0.5%, less than 0.1%, or less than 0.05% oxygen.

The gaseous phase of the second flushing medium can have a different composition from that of the gaseous phase of the first flushing medium. In particular, the gaseous phases of the first and second flushing medium can be different with respect to their oxygen content (e.g., in percent by volume). In some embodiments, the gaseous phase of the second flushing medium has an oxygen content that is less than the oxygen content of the gaseous phase of the first flushing medium. As a result, the catalyst can be exposed to a more oxidative atmosphere by contacting the first flushing medium, followed by exposure to a less oxidative or even inert atmosphere by contacting the second flushing medium. In particular embodiments, the gaseous phase of the first flushing medium comprises about 5% to about 20% oxygen by volume (e.g., air or similar gaseous composition having about 20% oxygen) and the gaseous phase of the second flushing medium is essentially free of oxygen (e.g., 99% or higher nitrogen).

The present method may include maintaining contact of the hydrogenation catalyst with the second flushing medium for a nitrogen-treatment duration to produce the regenerated hydrogenation catalyst. The nitrogen-treatment duration can be at least 30 minutes, including but not limited to at least 1 hour, at least 2 hours, at least 4 hours, at least 8 hours, at least 12 hours, at least 24 hours, or at least 48 hours. In some embodiments, the nitrogen-treatment duration is about 4 hours to about 24 hours, such as about 12 hours to about 24 hours. In some embodiments, the nitrogen-treatment duration is about 20 hours.

The air-treatment duration and the nitrogen-treatment duration in a given regeneration cycle can be adjusted and coordinated based on the other conditions of the regeneration cycle, such as temperature, pressure, and recycle rate of the flushing medium, in order to achieve desired regeneration results. For example, air-treatment duration can be 2-4 hours and the nitrogen-treatment duration can be about 20 hours in a regeneration cycle, in which the flushing medium is recycled at a recycle rate of 6-10 (such as a recycle rate of 8).

The contact of the first or the second flushing medium with the catalyst can be carried out, for example, by flowing the flushing medium across the surface of the catalyst. The contact can be maintained by controlling the flow rate of the flushing medium throughout the air-treatment duration and the nitrogen-treatment duration. As described herein, both the liquid and gaseous portion of the flushing medium can be recycled (as designed by the recycle rate described herein), and the present system can provide continuous flow of the recycled flushing medium to maintain the contact of the first or the second flushing medium with the catalyst. Typically, the contact of the gaseous component of the first flushing medium with the catalyst is stopped (e.g., switched off) before the contact of the gaseous component of the second flushing medium with the catalyst is started (e.g., switched on). When multiple regeneration cycles are performed, typically the contact of the second gaseous component of the flushing medium with the catalyst in the last cycle is stopped before the contact of the gaseous component of the first flushing medium with the catalyst in the next cycle is started. Thus, the present system allows for exposure of the catalyst to drastically different atmospheres (e.g., from primarily air to primarily nitrogen) in a single regenerating cycle or from one regeneration cycle to another regeneration cycle.

In some embodiments, the regeneration temperature is from 50° C. to 200° C. In some embodiments, the regeneration temperature is at least 50° C., or at least 60° C., or at least 70° C., or at least 80° C., or at least 90° C., or at least 100° C., or at least 110° C., or at least 120° C., or at least 130° C., to less than 140° C., or less than 150° C., or less than 160° C., or less than 170° C., or less than 180° C., or less than 190° C., or less than 200° C. In some embodiments, the regeneration cycle is performed at a temperature from about 70° C. to about 120° C.

In some embodiments, the regeneration pressure is from 20 psig to 300 psig. In some embodiments, the regeneration pressure is at least 20 psig, or at least 30 psig, or at least 40 psig, or at least 50 psig, or at least 60 psig, or at least 70 psig, or at least 80 psig, or at least 90 psig, or at least 100 psig, to less than 110 psig, or less than 125 psig, or less than 150 psig, or less than 200 psig, or less than 250 psig, or less than 300 psig. In some embodiments, the regeneration cycle is performed at a pressure from about 50 psig to about 200 psig.

In some embodiments, the flow of the gaseous phase of the flushing medium to the reactor can be stopped while the liquid phase of the flushing medium is continued. The duration of the extra liquid flushing occurs for at least 30 minutes, or at least 1 hour, or at least 2 hours, or at least 3 hours, or at least 4 hours, or at least 5 hours, at least 6 hours, or less than 24 hours, or less than 2 days, or less than 3 days, or less than 4 days, or less than 5 days, or less than 6 days, or less than one week, or longer.

In some embodiments, the oxygen content in the flushing medium (e.g., the gaseous phase of the first flushing medium) is selected based on the amount of hydrogenation catalyst 38 in the reactor 12. In some embodiments, the first flushing medium comprises an oxygen to catalyst flux ratio (O₂/cat/hr) from 0.1*10⁻³ to 100*10⁻³ (mol/g/hr). In some embodiments, the O₂/cat/hr flux ratio is at least 0.1*10⁻³ (mol/g/hr), such as at least 0.5*10⁻³ (mol/g/hr), or at least 1*10⁻³ (mol/g/hr). In some embodiments, the O₂/cat/hr flux ratio is less than 100*10⁻³ (mol/g/hr), such as less than 50*10⁻³ (mol/g/hr), less than 10*10⁻³ (mol/g/hr), or less than 5*10⁻³ (mol/g/hr).

In some embodiments, the water content in the first flushing medium is based on the amount of hydrogenation catalyst 38 in the reactor 12. In some embodiments, the first flushing medium comprises a water to catalyst flux ratio (H₂O/cat/hr) from 1 to 100 (g/g/hr). In some embodiments, the H₂O/cat/hr ratio of the first flushing medium is at least 1 (g/g/hr), such as at least 2 (g/g/hr), at least 5 (g/g/hr), or at least 10 (g/g/hr). In some embodiments, the H₂O/cat/hr ratio of the first flushing medium is less than 100 (g/g/hr), such as less than 50 (g/g/hr), less than 20 (g/g/hr), or less than 10 (g/g/hr).

In some embodiments, the second flushing medium comprises a nitrogen to catalyst flux ratio (N₂/cat/hr) from 0.1*10⁻³ to 100*10⁻³ (mols/w/hr). In some embodiments, the N₂/cat/hr flux ratio is at least 0.1*10⁻³ (mol/g/hr), such as at least 0.5*10⁻³ (mol/g/hr), or at least 1*10⁻³ (mol/g/hr). In some embodiments, the N₂/cat/hr flux ratio is less than 100*10⁻³ (mol/g/hr), such as less than 50*10⁻³ (mol/g/hr), less than 10*10⁻³ (mol/g/hr), or less than 5*10⁻³ (mol/g/hr).

In some embodiments, the water content in the second flushing medium is based on the amount of hydrogenation catalyst 38 in the reactor 12. In some embodiments, the second flushing medium comprises a water to catalyst flux ratio (H₂O/cat/hr) from 1 to 100 (g/g/hr). In some embodiments, the H₂O/cat/hr ratio of the second flushing medium is at least 1 (g/g/hr), such as at least 2 (g/g/hr), at least 5 (g/g/hr), or at least 10 (g/g/hr). In some embodiments, the H₂O/cat/hr ratio of the second flushing medium is less than 100 (g/g/hr), such as less than 50 (g/g/hr), less than 20 (g/g/hr), or less than 10 (g/g/hr).

The nitrogen content in the gaseous phase of the second flushing medium may be selected to provide a desired inert atmosphere in contrast to the oxidative atmosphere caused by the gaseous phase of the first flushing medium (e.g., at least 90% air by volume). In some embodiments, the second flushing medium comprises, measured at 25° C. and 1 atmospheric pressure, a gaseous phase comprising at least 99% nitrogen by volume.

The inclusion of water in the flushing medium offers various advantages. First, water acts as a heat sink in the flushing medium that allows improved control over the temperature of the reactor 12 relative to a flushing medium composed solely of gases. This improved heat control avoids the creation of hot spots that may burn away catalytic supports, such as carbon. Water is also a polar solvent that may facilitate the removal of certain impurities, such as ionic salts and other polar moieties. In addition, the inclusion of water in the flushing medium allows the hydrogenation catalyst 38 to remain wetted during regeneration. Flushing media composed solely of gases can dry out the catalyst, which can create cracks in the fixed bed and lead to an increase in replacement frequency.

In some embodiments, the flushing medium is substantially free or entirely free of hydrogen peroxide. As used herein, the term “substantially free of hydrogen peroxide” refers to less than 1%, or less than 0.5%, or less than 0.1%, or less than 0.05% hydrogen peroxide. In some embodiments, the flushing medium is substantially free or entirely free of hydrogen peroxide prior to entering the reactor 12.

In some embodiments, one or more the regeneration cycles are employed to produce the regenerated hydrogenation catalyst. For example, the method may include subjecting the fouled hydrogenation catalyst to one, two, three, four, or more regeneration cycles as described herein, and the conditions of one regeneration cycle (such as temperature, pressure, and duration) may be controlled independent of the corresponding conditions of another regeneration cycle. For example, a first regeneration cycle may be performed at a different temperature or a different pressure, or for a different duration than a second regeneration cycle.

Unlike typical catalyst regeneration processes, which operate in gas-phase conditions under temperatures in excess of 200° C. (e.g., decoking and desulphurization reactions), or utilize oxidants that degrade the catalyst's physical structure over time (e.g., H₂O₂-based regeneration), the present disclosure provides a method for regenerating a hydrogenation catalyst 38 with a flushing medium that operates under less severe conditions (e.g., temperatures of less than 200° C.). As reported in the co-pending U.S. patent application Ser. No. 17/891,093, a flushing medium comprising water, oxygen, and an inert/diluent gas at the specified regeneration pressures and temperatures is effective in restoring catalytic activity by removing impurities from the hydrogenation catalyst and maintaining the catalyst's structural integrity (e.g., total surface, pore size, pore volume). Surprisingly and remarkably, as shown in the present disclosure, the regeneration efficiency can be further improved by switching the gaseous phase of the flushing medium from air (higher oxygen or oxidative atmosphere) to nitrogen (lower oxygen or inert atmosphere), as more impurities (such as sulfur-containing impurities) can be removed the fouled catalyst during the nitrogen-treatment duration. By adapting to atmospheric switching between different gaseous phases, the system as described herein can regenerate fouled hydrogenation catalysts more efficiently at reduced cost. For example, the nitrogen-treatment duration can be adjusted according to the recycle rate of the flushing medium to reduce water consumption and improve removal of impurities (e.g., sulfur-containing impurities).

Furthermore, the present system allows for a “pulsed” regeneration process having multiple regeneration cycles, each cycle undergoing a switch between an oxidative atmosphere (e.g., a pulse of air-treatment) and an inert atmosphere (e.g., a pulse of nitrogen-treatment), thus providing more flexibility and efficiency in controlling the regeneration process. More significantly, the present “pulsed” regeneration system can be more effective in removing sulfur from a fouled catalyst than a system not utilizing air/nitrogen switching, where the feedstock contains higher than normal sulfur-containing impurities. Thus, the present system has unexpected and superior capability over the previous systems in regenerating catalysts used for hydrogenating feedstock with a high sulfur content.

In another aspect, the present disclosure provides a method for producing a regenerated hydrogenation catalyst from a fouled hydrogenation catalyst, the method comprising:
catalytically reacting a feedstock stream having at least one sulfur-containing impurity in the presence of a hydrogenation catalyst to produce the fouled hydrogenation catalyst, wherein the fouled hydrogenation catalyst comprises an amount of sulfur derived from the at least one sulfur-containing impurity of the feedstock stream, and
subjecting the fouled hydrogenation catalyst to a regeneration cycle to produce a regenerated hydrogenation catalyst, the regeneration cycle comprising:
   (a) contacting the catalyst with a first flushing medium for an air-treatment duration to produce an air-treated catalyst, wherein the first flushing medium comprises, measured at 25° C. and 1 atmospheric pressure, liquid water and a gaseous phase comprising oxygen, and
   (b) contacting the air-treated catalyst with a second flushing medium for a nitrogen-treatment duration to produce a nitrogen-treated catalyst, wherein the second flushing medium comprises, measured at 25° C. and 1 atmospheric pressure, liquid water and a gaseous phase comprising at least 90% nitrogen by volume,
wherein the amount of sulfur in the nitrogen-treated hydrogenation catalyst is reduced relative to the fouled hydrogenation catalyst.

The fouled hydrogenation catalyst can be, for example, a hydrogenation catalyst that has been exposed to a feedstock solution under the specified hydrogenation conditions (e.g., temperatures, pressures, concentrations of feedstock) described herein for a period of time (e.g., at least 1 day, at least 2 days, at least 3 days, at least 4 days, at least 5 days, at least 6 days, at least one week, at least two weeks, at least three weeks, at least one month, at least six months, at least one year). The fouled hydrogenation catalyst can be produced by exposing a fresh catalyst that has never been exposed to the feedstock solution at the specified hydrogenation conditions, or by exposing a freshly regenerated catalyst to the specified hydrogenation conditions.

Suitable first and second flushing media include those described above. In some embodiments, the gaseous phase of the first flushing medium comprises about 0.1% to about 30% oxygen by volume. The gaseous phase of the first flushing medium can further comprise an inert gas, such as nitrogen, argon, helium, neon, krypton, xenon, radon, carbon dioxide, or a combination thereof. In some embodiments, the gaseous phase of the first flushing medium comprises at least 90% air by volume. In some embodiments, the gaseous phase of the second flushing medium comprises at least 99% nitrogen by volume. In some embodiments, the gaseous phase of the second flushing medium is essentially free of oxygen. In some embodiment, the method comprises subjecting the nitrogen-treated catalyst to a successive set of the operations (a) and (b) of the regeneration cycle, each with a respective treatment duration, to produce the regenerated hydrogenation catalyst. For example, the method may include subjecting the nitrogen-treated catalyst to one, two, three, four, or more successive regeneration cycles as described herein. Advantageously, the conditions of each of the regeneration cycles (such as temperature, pressure, and duration) may be independently controlled. Also, the air-treatment duration and the nitrogen-treatment duration in each regeneration cycle can be coordinated to improve regeneration efficiency, based on the sulfur content in the feedstock, the amount of sulfur in the fouled catalyst, and recycle rate of the flushing medium.

In some embodiments, the air-treatment duration in one or more of the regeneration cycles is at least 30 minutes, such as about 2-4 hours.

In some embodiments, the nitrogen-treatment duration in one or more of the regeneration cycles is at least 30 minutes or at least 4 hours, such as at about 12-24 hours.

In some embodiments, the amount of sulfur in the nitrogen-treated hydrogenation catalyst or the regenerated hydrogenation catalyst is reduced by at least 5% relative to the fouled hydrogenation catalyst. The reduction can be at least a 10% reduction, at least a 15% reduction, at least a 20% reduction, at least a 30% reduction, at least a 40% reduction, at least a 50% reduction, or at least a 60% reduction relative to the sulfur content of the fouled hydrogenation catalyst.

In some embodiments, the air-treatment duration is at least 30 minutes, the nitrogen-treatment duration is at least 4 hours, and the amount of sulfur in the regenerated hydrogenation catalyst is reduced by at least 5% relative to the fouled hydrogenation catalyst. In some embodiments, multiple regeneration cycles (e.g., a total of two, three, four, five, six, seven, eight, night, or ten cycles) are performed in the present method, wherein at least one of the cycles includes an air-treatment duration of at least 30 minutes and an nitrogen-treatment duration of at least 4 hours, and wherein the amount of sulfur in the regenerated hydrogenation catalyst is reduced by at least 5% relative to the fouled hydrogenation catalyst.

In some embodiments, the regeneration cycle is performed at a temperature from about 70° C. to about 120° C. In some embodiments, multiple regeneration cycles (e.g., a total of two, three, four, five, six, seven, eight, night, or ten cycles) are performed in the present method and at least one of the multiple regeneration cycles is performed at a temperature from about 70° C. to about 120° C.

In some embodiments, the regeneration cycle is performed at a pressure from about 50 psig to about 200 psig. In some embodiments, multiple regeneration cycles (e.g., a total of two, three, four, five, six, seven, eight, night, or ten cycles) are performed in the present method and at least one of the multiple regeneration cycles is performed at a pressure from about 50 psig to about 200 psig.

The present method can be particularly effective in in regenerating catalysts used for hydrogenating feedstock with a higher than normal level of sulfur-containing impurities. In some embodiments, the feedstock stream has an amount of sulfur of at least 0.1 ppm, including but not limited to at least 0.2 ppm, at least 0.3 ppm, at least 0.4 ppm, at least 0.5 ppm, at least 1.0 ppm, at least 2.0 ppm, at least 5.0 ppm, at least 10.0 ppm, at least 20 ppm, at least 30 ppm, at least 40 ppm, at least 50 ppm, and at least 100 ppm. In some embodiments, the feedstock stream has an amount of sulfur of at least 0.5 ppm, such as at least 1.0 ppm or at least 5 ppm.

In some embodiments, the amount of sulfur removed from the catalyst is measured to monitor the progress of the regeneration process. Based on this information, the conditions for further regeneration cycles (such as temperature, pressure, air-treatment duration, and nitrogen-treatment duration) can be adjusted to improve efficiency. In some embodiments, the present method further comprises: measuring a first sulfur content of the first flushing medium after contacting the catalyst in (a) and/or measuring a second sulfur content of the second flushing medium after contacting the catalyst in (b); and subjecting the nitrogen-treated catalyst to a successive set of the operations (a) and (b), each with a respective treatment duration. The sulfur content in the flushing medium can be measured by any suitable analytic method. For example, the sulfur content can be measured by measuring conductivity and/or pH of the first flushing medium or the second flushing medium or by inductively coupled plasma (ICP) methods.

In some embodiments, the regenerated hydrogenation catalysts exhibit excellent retention in catalytic activity after regenerative treatment. As used herein, the term “retain,” “retaining,” or “retention,” with respect to a reference value, includes both partial and increased values relative to the reference value. For example, a specified parameter (e.g., conversion of a regenerated catalyst) can retain less than 100% or more than 100% of a reference parameter (e.g., conversion of a fresh catalyst or a fouled catalyst from which the regenerated catalyst is produced).

Typically, the fouled catalyst has lower catalytic capacity (e.g., as measured by the conversion value) than the fresh catalyst. The catalytic capacity of a fouled catalyst may be increased by the regeneration method as described herein to a level close to that of the fresh (or freshly regenerated) catalyst from which the fouled catalyst is produced. That is, the regeneration method herein may be used to restore the catalytic capacity of a fouled catalyst back to the level of the fresh (or freshly regenerated) catalyst. The conversion value as describe herein (as a measurement of catalytic capacity) of a regenerated catalyst, or the conversion value of a fouled catalyst from which the regenerated catalyst is produced, may be compared to that of a fresh catalyst. For example, the conversion value of a fouled catalyst may be about 50%, about 60%, about 70%, about 80%, or about 90% of the conversion value of a fresh catalyst. For example, the conversion value of a regenerated catalyst may be about 70%, about 80%, about 90%, about 95%, about 99%, about 100%, or about 110% the catalytic capacity of a fresh catalyst. In some embodiments, the conversion value of a regenerated catalyst is at least 5%, at least 10%, at least 20%, at least 50%, at least 70%, at least 90%, or at least 100% higher than that of a fouled catalyst. In some embodiments, regenerated catalyst retains at least 100%, at least 105%, at least 110%, at least 120%, at least 150%, at least 170%, at least 190%, or at least 200% of the conversion value of a fouled catalyst. As an example, the conversion value of a fresh catalyst is 0.96 and the conversion value of a fouled catalyst is 0.70 (or 73% of the fresh catalyst). After regeneration, the conversion value of the regenerated catalyst is 0.94 (or 98% of the fresh catalyst). In this example, the regenerated catalyst retains 134% of the conversion of the fouled catalyst (or the conversion value of a regenerated catalyst is 34% higher than that of the fouled catalyst).

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms. As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter. As used herein, the singular forms “a”, “an”, and “the” include plural embodiments unless the context clearly dictates otherwise. The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

The present invention has been described in terms of one or more preferred embodiments, and it should be appreciated that many equivalents, alternatives, variations, and modifications, aside from those expressly stated, are possible and within the scope of the invention.

### EXAMPLES

### Example 1

Regeneration tests on hydrogenation catalysts having impurities were conducted using a flushing medium that included a liquid phase comprising water and a vapor phase comprising nitrogen and oxygen. The process was termed “wet air oxidation regeneration” (WAOR). Fresh Ru/C hydrogenation catalysts were deactivated by hydrogenating dextrose monohydrate or corn syrup feeds. The hydrogenation reactions were operated on stream for multiple days until the hydrogenation catalysts were deactivated. WAOR experiments were performed on deactivated hydrogenation catalysts. Conditions for a standard WAOR process were as follows: 100% air, 100-110° C. inlet temperature, 100 psig reactor pressure, with water and air flowed for 24 hours. After 24 hours, water flow continued but the system was purged with N₂ to remove any traces of oxygen before hydrogen was introduced to reduce the catalyst. In various studies conducted herein, an oxygen level of 1% (e.g., 5% air, 95% nitrogen)-20% (e.g., 100% air) in the vapor phase of the flushing medium was used with similar results. Nitrogen as used herein was supplied as a high purity gas (e.g., 99% or higher).

While the standard WAOR regeneration may be useful for recovering catalyst activity with majority of corn syrups in tested process, it was insufficient for catalyst recovery when high-sulfur corn syrup feeds were used, even with extended time using air. The sulfur impurities deposited on the catalyst may be removed in a regeneration process, the result of which can be measured by the amount of sulfur in the effluent liquid phase (water) of the flushing medium (e.g., by weight or by wt % relative to the sulfur in the feed). Remarkably, it was observed in some experiments that the bulk of sulfur was removed during the WAOR process only after switching to N₂ purge, and minimal or no sulfur was removed under air flow. Further, results from WAOR experiments with different air flow duration (e.g., 24 hours vs. 4 hours) were compared, which showed that the sulfur content in the effluent water peaked at approximately the same time after switching to N₂ regardless of the air flow duration (e.g., 2-6 hours after switching to N₂ following either 4 hours or 24 hours of air flow). More significantly, similar amounts of sulfur were removed (as measured by wt % relative to the sulfur in the feed) in these experiments with different air flow duration (17% removed with 24 hours air flow vs. 14% removed with 4 hours air flow). These results suggest that a predominant amount of sulfur is removed from the deactivated catalyst after N₂ flow is introduced rather than during air flow.

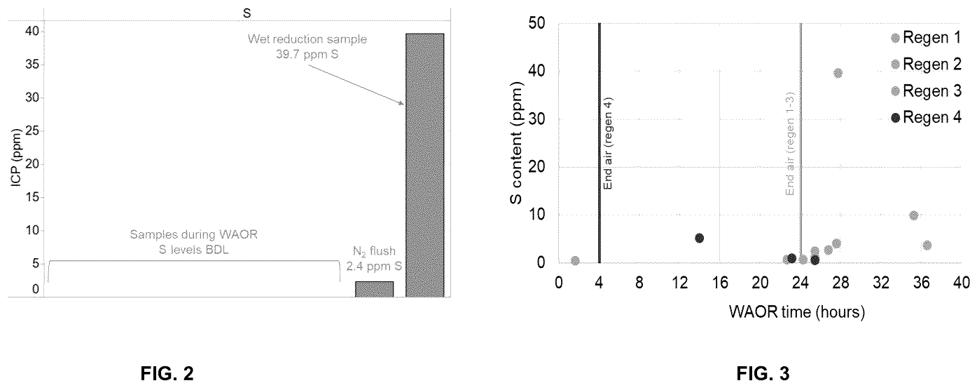

Further regeneration studies were performed on a 2 wt % Ru/C catalyst after approximately 50 days reaction with feed stream. Samples of the reactor effluent were taken during the course of the regeneration, after nitrogen was introduced to flush the reactor of oxygen, and during the wet reduction. No sulfur was detected during the regeneration under air flow, but significant levels were observed after the atmosphere in the reactor was switched to N₂ and then H₂ (FIG. 2). These results indicated that changing the reactor atmosphere may affect the removal of sulfur from the catalyst.

Similar results were observed from a regeneration in additional studies, where the sulfur content of the effluent was tracked every four hours (Table 1). The maximum sulfur content observed during air flow was at the very end of having air in the system (6.1 ppm sulfur after 24 hours). However, the first sample after switching to N₂ showed a sulfur content of 24 ppm (Table 1), demonstrating a significant increase in sulfur removal after switching from air to N₂. This result again showed that removal of sulfur was linked to the change in reactor atmosphere.

TABLE 1
Sulfur content of reactor effluent for a regeneration process.

| Description | Sulfur (ppm) |
|---|---|
| 4 hours after starting air flow | BDL |
| 8 hours after starting air flow | BDL |
| 12 hours after starting air flow | 1.3 |
| 16 hours after starting air flow | 2.7 |
| 20 hours after starting air flow | 4.3 |
| 24 hours after starting air flow | 6.1 |
| 4 hours after switching to N2 | 24 |
| 8 hours after switching to N2 | 22.9 |

In a further study, instead of running air through the reactor during the regeneration for 24 hours, air was only supplied for 4 hours. The first ICP samples were only collected after air flow had been stopped for a few hours, but it appeared that the sulfur levels in the effluent were already past their peak and dropping. These data indicate that sulfur content peaked after switching from air to N₂ (FIG. 3).

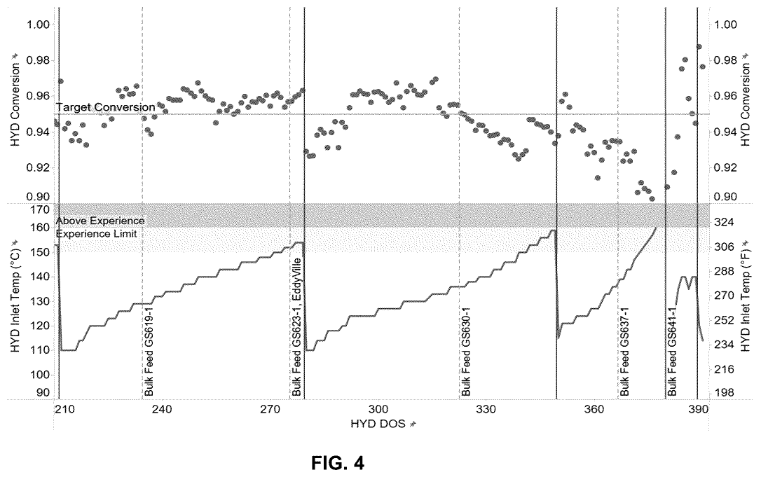

With normal corn syrup feed and standard WAOR regeneration procedure, the catalyst may have a cycle time of approximately 50-70 days, after which the catalyst needs to be regenerated to maintain sufficient conversion of the glucose to sorbitol. However, when corn syrup with higher than normal levels of sulfur (e.g., 0.5 ppm or higher) was used as feed, 3- to 4-fold higher rates of deactivation of the catalyst were observed. The high loading of sulfur impurities on the catalyst necessitated an earlier-than-normal WAOR. A first regeneration was needed only after 30 days with these feeds (FIG. 4, regeneration at around 380 days). Even after this regeneration, poor catalyst activity was observed, and a second regeneration was required only 10 days later (FIG. 4, regeneration at around 390 days). Significant levels of sulfur were observed in the effluent from these regenerations.

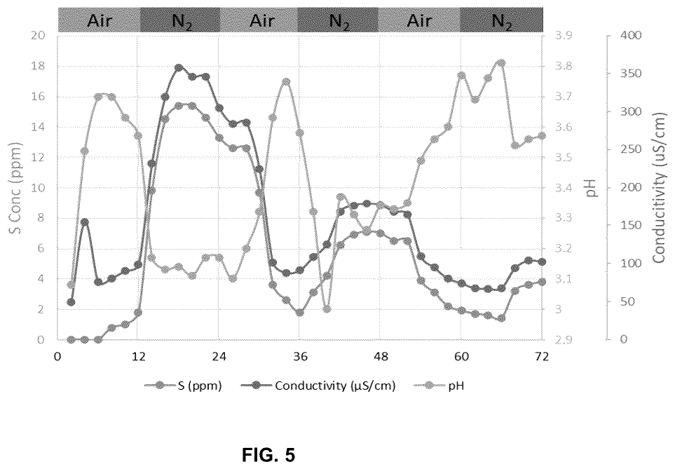

Based on the observation that sulfur was primarily removed from the catalyst after switching from air flow to nitrogen, as well as the hypothesis that the short cycle time at 380 days on stream was due to insufficient sulfur removal from the catalyst, a new “pulsed” regeneration procedure was developed. The “pulsed” procedure was designed to use the same reactor temperature, pressure, and gas and liquid flow rates, but switch from air to nitrogen after only 12 hours. Following 12 hours of purging with nitrogen, air was re-introduced. Following another 12 hours of air flow, nitrogen was introduced, and then one final cycle of 12 hours air/12 hours nitrogen was performed. Samples of the aqueous effluent were collected periodically, and the pH, conductivity, and sulfur content were analyzed (FIG. 5). Consistent with previous observations, the sulfur levels did not spike until after the atmosphere had been switched to N₂. In parallel with the spike in sulfur levels, the conductivity rapidly increased to 350 uS/cm, and the pH dropped from about 3.7 to 3.2. Each of these effects are consistent with the sulfur being removed from the catalyst as an acidic substance (e.g., H₂SO₄). Following the switch back to air, the sulfur levels and conductivity decreased, while the pH increased. A second N₂ purge again showed an increase in sulfur content and conductivity, showing that this second air/N₂ cycle removed additional sulfur from the catalyst, though in an amount less than that of the first cycle. The third cycle further removed significant amount of sulfur from the catalyst (although the amount was less than those of the first and second cycles).

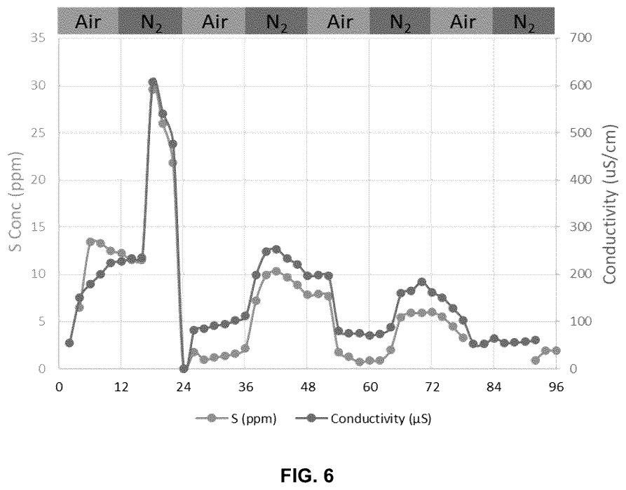

After this pulsed regeneration, feedstock was again introduced to the reactor after reduction. This feed also contained higher levels of sulfur. Remarkably, catalytic activity was restored to start-of-run levels, showing that the pulsed regeneration process was more effective at removing high levels of sulfur poisoning from the catalyst than the previous, standard WAOR regeneration process. The catalyst was then on stream for 19 days, when another regeneration was required due to the high levels of sulfur poisons in the feed. Similar to previous regeneration processes, sulfur levels spiked after switching to N₂, which was also consistent with conductivity data (FIG. 6). Due to the case of measuring conductivity, the rapidity with which a sample can be analyzed, and the potential for on-line measurement, conductivity data were used as a reliable marker for sulfur content. This regeneration process showed similar conductivity levels in the third pulse as the second pulse, so a fourth pulse was conducted.

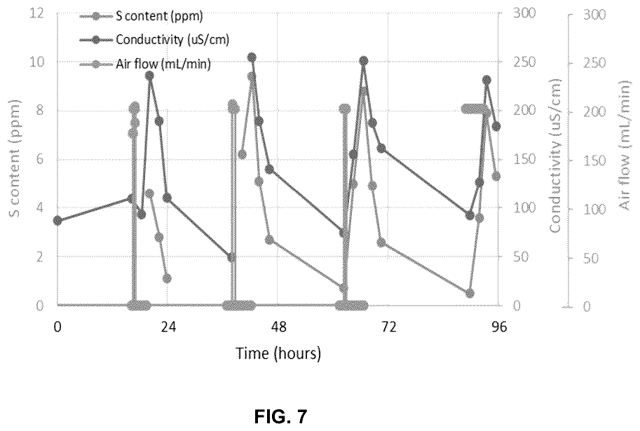

The pulsed regeneration procedure was further developed. On a different plant, an air purge of only 30 minutes was attempted, down from 12 hours. This change would significantly decrease the regeneration time. These short pulses were still effective at removing sulfur from the catalyst (FIG. 7). Each of the four pulses showed a similar magnitude of conductivity increase and sulfur content in the effluent, suggesting incomplete sulfur oxidation during each pulse. Pulse duration in these processes may be further designed to maximize sulfur removal in short time.

In summary, a pulsed regeneration process with air/N₂ atmospheric switching was implemented for hydrogenation catalysts, and the performance of the catalyst regenerated by the present method returned much closer to start-of-run activity, compared to the activity of the catalyst regenerated by the previous non-pulsed regeneration. Significant amounts of sulfur were removed during the nitrogen treatment, and high levels of sulfur can be removed by multiple regeneration cycles (pulses). For example, the level of sulfur can remain high in the effluent of the second and third regeneration cycles (pulses). The conductivity and pH of the effluent was used to track the amount of sulfur removed from the catalyst. In some tests, a pulsed regeneration process was conducted using 30 minutes of air flow and 23.5 hours of nitrogen flow in each pulse, which remained effective in removing significant amount of sulfur from the catalyst.

For reasons of completeness, various aspects of the invention are set out in the following numbered clauses:

Clause 1. A method for hydrogenating biomass, the method comprising:
catalytically reacting a feedstock stream comprising water and an oxygenated hydrocarbon (C₂₊O₁₊) with hydrogen in the presence of a hydrogenation catalyst for a hydrogenation duration to produce a first hydrogenated product stream and a fouled hydrogenation catalyst;
subjecting the fouled hydrogenation catalyst to a regeneration cycle to produce a regenerated hydrogenation catalyst, the regeneration cycle comprising:
   (a) contacting the catalyst with a first flushing medium for an air-treatment duration to produce an air-treated catalyst, wherein the first flushing medium comprises, measured at 25° C. and 1 atmospheric pressure, liquid water and a gaseous phase comprising oxygen, and
   (b) contacting the air-treated catalyst with a second flushing medium for a nitrogen-treatment duration to produce the regenerated hydrogenation catalyst, wherein the second flushing medium comprises, measured at 25° C. and 1 atmospheric pressure, liquid water and a gaseous phase comprising at least 90% nitrogen by volume; and
catalytically reacting the feedstock stream with hydrogen in the presence of the regenerated hydrogenation catalyst to further produce a second hydrogenated product stream.

Clause 2. The method of clause 1, the air-treatment duration is at least 30 minutes.

Clause 3. The method of any one of clauses 1-2, the nitrogen-treatment duration is at least 30 minutes.

Clause 4. The method of any one of clauses 1-3, wherein the regeneration cycle is performed at a temperature from about 70° C. to about 120° C.

Clause 5. The method of any one of clauses 1-4, wherein regeneration cycle is performed at a pressure from about 50 psig to about 200 psig.

Clause 6. The method of any one of clauses 1-5, wherein the first flushing medium comprises an oxygen to catalyst flux ratio (O₂/cat/hr) from 0.1*10⁻³ to 100*10⁻³ (mols/g/hr).

Clauses 7. The method of any one of clauses 1-6, wherein the first flushing medium and/or the second flushing medium comprise a water to catalyst flux ratio (H₂O/cat/hr) from 1 to 100 (g/g/hr).

Clause 8. The method of any one of clauses 1-7, wherein the second flushing medium comprises a nitrogen to catalyst flux ratio (N₂/cat/hr) from 0.1*10⁻³ to 100*10⁻³ (mols/g/hr).

Clause 9. The method of any one of clauses 1-8, wherein the gaseous phase of the first flushing medium comprises about 0.1% to about 30% oxygen by volume.

Clause 10. The method of any one of clauses 1-9, wherein the gaseous phase of the first flushing medium further comprises an inert gas selected from the group consisting of nitrogen, argon, helium, neon, krypton, xenon, radon, carbon dioxide, and a combination thereof.

Clause 11. The method of any one of clauses 1-10, wherein the gaseous phase of the first flushing medium comprises at least 90% air by volume.

Clause 12. The method of any one of clauses 1-11, wherein the gaseous phase of the second flushing medium comprises at least 99% nitrogen by volume.

Clause 13. The method of any one of clauses 1-12, wherein the gaseous phase of the second flushing medium is essentially free of oxygen.

Clauses 14. The method of any one of clauses 1-13, wherein the oxygenated hydrocarbon is a saccharide.

Clauses 15. The method of any one of clauses 1-14, wherein the hydrogenation catalyst comprises a support and an active metal.

Clause 16. The method of claim 15, wherein the hydrogenation catalyst is ruthenium on carbon (Ru/C).

Clause 17. A method for producing a regenerated hydrogenation catalyst from a fouled hydrogenation catalyst, the method comprising:
catalytically reacting a feedstock stream having at least one sulfur-containing impurity in the presence of a hydrogenation catalyst to produce the fouled hydrogenation catalyst, wherein the fouled hydrogenation catalyst comprises an amount of sulfur derived from the at least one sulfur-containing impurity of the feedstock stream, and
subjecting the fouled hydrogenation catalyst to a regeneration cycle to produce a regenerated hydrogenation catalyst, the regeneration cycle comprising:
   (a) contacting the catalyst with a first flushing medium for an air-treatment duration to produce an air-treated catalyst, wherein the first flushing medium comprises, measured at 25° C. and 1 atmospheric pressure, liquid water and a gaseous phase comprising oxygen, and
   (b) contacting the air-treated catalyst with a second flushing medium for a nitrogen-treatment duration to produce a nitrogen-treated catalyst, wherein the second flushing medium comprises, measured at 25° C. and 1 atmospheric pressure, liquid water and a gaseous phase comprising at least 90% nitrogen by volume,
wherein the amount of sulfur in the nitrogen-treated hydrogenation catalyst is reduced relative to the fouled hydrogenation catalyst.

Clause 18. The method of clause 17, comprising subjecting the nitrogen-treated catalyst to a successive set of the operations (a) and (b) of the regeneration cycle, each with a respective treatment duration, to produce the regenerated hydrogenation catalyst.

Clause 19. The method of any one of clauses 17-18, the air-treatment duration is at least 30 minutes.

Clause 20. The method of any one of clauses 17-19, the nitrogen-treatment duration is at least 30 minutes.

Clause 21. The method of any one of clauses 17-20, wherein the air-treatment duration is at least 30 minutes, wherein the nitrogen-treatment duration is at least 4 hours, and wherein the amount of sulfur in the regenerated hydrogenation catalyst is reduced by at least 5% relative to the fouled hydrogenation catalyst.

Clauses 22. The method of any one of clauses 17-21, wherein the regeneration cycle is performed at a temperature from about 70° C. to about 120° C.

Clause 23. The method of any one of clauses 17-22, wherein the regeneration cycle is performed at a pressure from about 50 psig to about 200 psig.

Clause 24. The method of any one of clauses 17-23, wherein the feedstock stream has an amount of sulfur of at least 0.1 ppm.

Clause 25. The method of any one of clauses 17-24, wherein the gaseous phase of the first flushing medium comprises about 0.1% to about 30% oxygen by volume.

Clause 26. The method of any one of clauses 17-25, wherein the gaseous phase of the first flushing medium further comprises an inert gas selected from the group consisting of nitrogen, argon, helium, neon, krypton, xenon, radon, carbon dioxide, and a combination thereof.

Clause 27. The method of any one of clauses 17-26, wherein the gaseous phase of the first flushing medium comprises at least 90% air by volume.

Clause 28. The method of any one of clauses 17-27, wherein the gaseous phase of the second flushing medium comprises at least 99% nitrogen by volume.

Clause 29. The method of any one of clauses 17-28, wherein the gaseous phase of the second flushing medium is essentially free of oxygen.

1. A method for hydrogenating biomass, the method comprising:
catalytically reacting a feedstock stream comprising water and an oxygenated hydrocarbon (C₂₊O₁₊) with hydrogen in the presence of a hydrogenation catalyst for a hydrogenation duration to produce a first hydrogenated product stream and a fouled hydrogenation catalyst;
subjecting the fouled hydrogenation catalyst to a regeneration cycle to produce a regenerated hydrogenation catalyst, the regeneration cycle comprising:
(a) contacting the catalyst with a first flushing medium for an air-treatment duration to produce an air-treated catalyst, wherein the first flushing medium comprises, measured at 25° C. and 1 atmospheric pressure, liquid water and a gaseous phase comprising oxygen, and
(b) contacting the air-treated catalyst with a second flushing medium for a nitrogen-treatment duration to produce the regenerated hydrogenation catalyst, wherein the second flushing medium comprises, measured at 25° C. and 1 atmospheric pressure, liquid water and a gaseous phase comprising at least 90% nitrogen by volume; and
catalytically reacting the feedstock stream with hydrogen in the presence of the regenerated hydrogenation catalyst to further produce a second hydrogenated product stream.

2. The method of claim 1, the air-treatment duration is at least 30 minutes.

3. The method of claim 1, the nitrogen-treatment duration is at least 30 minutes.

4. The method of claim 1, wherein the regeneration cycle is performed at a temperature from about 70° C. to about 120° C.

5. The method of claim 1, wherein regeneration cycle is performed at a pressure from about 50 psig to about 200 psig.

6. The method of claim 1, wherein the first flushing medium comprises an oxygen to catalyst flux ratio (O₂/cat/hr) from 0.1*10⁻³ to 100*10⁻³ (mols/g/hr).

7. The method of claim 1, wherein the first flushing medium and/or the second flushing medium comprise a water to catalyst flux ratio (H₂O/cat/hr) from 1 to 100 (g/g/hr).

8. The method of claim 1, wherein the second flushing medium comprises a nitrogen to catalyst flux ratio (N₂/cat/hr) from 0.1*10⁻³ to 100*10⁻³ (mols/g/hr).

9. The method of claim 1, wherein the gaseous phase of the first flushing medium comprises about 0.1% to about 30% oxygen by volume.

10. The method of claim 1, wherein the gaseous phase of the first flushing medium further comprises an inert gas selected from the group consisting of nitrogen, argon, helium, neon, krypton, xenon, radon, carbon dioxide, and a combination thereof.

11. The method of claim 1, wherein the gaseous phase of the first flushing medium comprises at least 90% air by volume.

12. The method of claim 1, wherein the gaseous phase of the second flushing medium comprises at least 99% nitrogen by volume.

13. The method of claim 1, wherein the gaseous phase of the second flushing medium is essentially free of oxygen.

14. The method of claim 1, wherein the oxygenated hydrocarbon is a saccharide.

15. The method of claim 1, wherein the hydrogenation catalyst comprises a support and an active metal.

16. The method of claim 15, wherein the hydrogenation catalyst is ruthenium on carbon (Ru/C).

17. A method for producing a regenerated hydrogenation catalyst from a fouled hydrogenation catalyst, the method comprising:
catalytically reacting a feedstock stream having at least one sulfur-containing impurity in the presence of a hydrogenation catalyst to produce the fouled hydrogenation catalyst, wherein the fouled hydrogenation catalyst comprises an amount of sulfur derived from the at least one sulfur-containing impurity of the feedstock stream, and
subjecting the fouled hydrogenation catalyst to a regeneration cycle to produce a regenerated hydrogenation catalyst, the regeneration cycle comprising:
(a) contacting the catalyst with a first flushing medium for an air-treatment duration to produce an air-treated catalyst, wherein the first flushing medium comprises, measured at 25° C. and 1 atmospheric pressure, liquid water and a gaseous phase comprising oxygen, and
(b) contacting the air-treated catalyst with a second flushing medium for a nitrogen-treatment duration to produce a nitrogen-treated catalyst, wherein the second flushing medium comprises, measured at 25° C. and 1 atmospheric pressure, liquid water and a gaseous phase comprising at least 90% nitrogen by volume,
wherein the amount of sulfur in the nitrogen-treated hydrogenation catalyst is reduced relative to the fouled hydrogenation catalyst.

18. The method of claim 17, comprising subjecting the nitrogen-treated catalyst to a successive set of the operations (a) and (b) of the regeneration cycle, each with a respective treatment duration, to produce the regenerated hydrogenation catalyst.

19-23. (canceled)

24. The method of claim 17, wherein the feedstock stream has an amount of sulfur of at least 0.1 ppm.

25-26. (canceled)

27. The method of claim 17, wherein the gaseous phase of the first flushing medium comprises at least 90% air by volume.

28-29. (canceled)
